(19) United States
(12) Patent Application Publication
(10) Pub. No.: US 2015/0238113 A1
(43) Pub. Date: Aug. 27, 2015

(54) APPARATUS AND METHOD FOR BREAST CANCER DETECTION BASED ON THE MEASURED MICROWAVE SIGNAL DECOMPOSITION

(71) Applicant: Electronics and Telecommunications Research Institute, Daejeon (KR)

(72) Inventors: Seong Ho SON, Daejeon (KR); Nikolai SIMONOV, Daejeon (KR); Soon Ik JEON, Daejeon (KR); Hyung Do CHOI, Daejeon (KR)

(21) Appl. No.: 14/631,407

(22) Filed: Feb. 25, 2015

(30) Foreign Application Priority Data
Feb. 26, 2014 (KR) 10-2014-0022389

(51) Int. Cl. A61B 5/05; A61B 5/00

(52) U.S. Cl. CPC A61B 5/0507; A61B 5/4312; A61B 5/726

(57) ABSTRACT

Disclosed is an apparatus for breast cancer detection using a microwave, which decomposes a microwave signal measured on the circumference of a breast for each of a plurality of frequency components and detects a breast cancer based on the microwave signals decomposed for each of the plurality of frequency components to increase accuracy of diagnosis of the breast cancer.

This application claims the benefit of priority of Korean Patent Application No. 10-2014-0022389 filed on Feb. 26, 2014, which is incorporated by reference in its entirety herein.

### BACKGROUND OF THE INVENTION

1. Field of the Invention

The present invention relates to an apparatus and a method for breast cancer detection using a microwave, and more particularly, to processing and analysis and imaging techniques of a measured microwave signal.

2. Discussion of the Related Art

At present, a breast cancer detecting apparatus using a microwave measures an amplitude and a phase of a microwave signal scattered by a breast according to a series of processes of transmitting and receiving a microwave through a plurality of transmission and reception antennas deployed on the circumference of the breast which is an object to be cancer-screened and finds an optimal breast tomography image which a difference (for example, a phase difference or an amplitude difference) between the measured microwave signal and a microwave signal calculated based on an estimated tomography image is minimum and displays the found optimal breast tomography image on a screen. The optimal breast tomography image shows a restored dielectric or conductivity value of an internal organization of the breast.

Meanwhile, a small-sized tumor of 5 mm or less needs to be detected for an early diagnosis of a breast cancer. However, there is a limit in detecting the small-sized tumor by the related art.

### SUMMARY OF THE INVENTION

The present invention has been made in an effort to provide an apparatus for breast cancer detection using a microwave signal, which processes and analyzes a measured microwave signal so as to read existence of a small-sized tumor.

The present invention has also been made in an effort to provide a method for processing a microwave signal, which restores a high-precision tomography image of a breast.

An embodiment of the present invention provides an apparatus for apparatus for breast cancer detection using a microwave signal, the apparatus including: a microwave signal decomposing unit configured to decompose a microwave signal measured along a circumference of a breast into a plurality of frequency components; and a beast cancer determining unit configured to determining the breast cancer based on the microwave signal decomposed into the plurality of frequency components.

Further, the microwave signal decomposing unit may decompose the measured microwave signal into the plurality of frequency components by performing wavelet transform with respect to the measured microwave signal.

The breast cancer determination unit may determine that the breast cancer exists at a portion having a value of a threshold value or more in the microwave signal decomposed into the plurality of frequency components.

Further, the apparatus may further include a weight allocating unit configured to apply at least one weight to the microwave signal decomposed into the plurality of frequency components.

Another embodiment of the present invention provides an apparatus for apparatus for breast cancer detection using a microwave signal, the apparatus including: a microwave signal measuring unit configured to measure the microwave signal along the circumference of the breast; a microwave signal decomposing unit including a first signal decomposing unit decomposing the microwave signal measured from the microwave signal measuring unit into a plurality of frequency components to generate a plurality of first microwave signals and a second signal decomposing unit decomposing a pre-calculated microwave signal into a plurality of frequency components to generate a plurality of second microwave signals; a weight allocating unit configured to calculate differences between the plurality of first microwave signals and the second microwave signals to generate a plurality of differential microwave signals and apply at least one weight to the plurality of differential microwave signals to generate one or more weight microwave signals; and an image restoring unit configured to restore a tomography of the breast by using the microwave signal provided from the weight allocating unit.

Further, the weight allocating unit may generate the weight microwave signals by applying the weights to all of the plurality of differential microwave signals, and the image restoring unit may generate a synthesized microwave signal by combining the weight microwave signals and updates the image so that the synthesized microwave signal is the minimum.

Further, the image restoring unit may perform the image restoration N times in sequence, and during the N-th image restoration, applies first to N-th weights to the plurality of differential microwave signals, performs the image restoration by setting the remaining weights to 0, and sets the N−1-th image restoration result as an initial condition of the N-th image restoration result.

The apparatus may further include a breast cancer determining unit configured to determine that the breast cancer exists at a portion having a value of a threshold value or more in the plurality of first microwave signals.

The breast cancer determining unit may further include a look-up table that pre-store the threshold value that is determined as the tumor in the breast, based on the values obtained by decomposing the microwave signal measured in the breast without the tumor for each frequency component.

Another embodiment of the present invention provides a method for breast cancer detection using a microwave signal, including: a microwave signal decomposition step of decomposing a microwave signal measured on the circumference of a breast for each of a plurality of frequency components; and a breast cancer determining step of determining a breast cancer based on the microwave signal decomposed for each of the plurality of frequency components.

In the microwave signal decomposition step, the measured microwave signal is wavelet-converted to decompose the measured microwave signal for each of the plurality of frequency components.

In the step of determining the breast cancer, it may be determined that the breast cancer exists at a portion showing a threshold value or more among the microwave signals decomposed for each of the plurality of frequency components.

The method may further include a weight allocating step of applying at least one weight to the microwave signal decomposed after the microwave signal decomposition step.

Yet another embodiment of the present invention provides a method for breast cancer detection using a microwave signal, including: a microwave signal decomposing step including a first signal decomposing step of decomposing the measured microwave signal for each of the plurality of frequency components to generate a plurality of first microwave signals and a second signal decomposing step of decomposing a precalculated microwave signal for each of the plurality of frequency components to generate a plurality of second microwave signals; a differential microwave generating step of acquiring differences of the plurality of first microwave signals having the frequency components and the plurality of first microwave to generate a plurality of differential microwave signals; a weight applying step of applying weights to at least one of the plurality of differential microwave signals to generate at least one weight microwave signal; and an image restoring step of restoring the tomography image of the breast by using at least one weight microwave signal provided from the weight applying step.

The precalculated microwave signal may be a microwave signal calculated in an estimated image in an initial step or an intermediate step.

In the weight applying step, the weights are applied to all of the plurality of differential microwave signals to generate the plurality of weight microwave signal, and in the image restoring step, the plurality of weight microwave signals are combined to generate a synthesized microwave signal and update an image so that the synthesized microwave signal is minimum.

In the image restoring step, image restoration may be sequentially performed N times, and in N-th image restoration, first to N-th weights may be applied to the plurality of differential microwave signals and the image restoration may be performed by setting a remaining weight to 0, and an N−1-th image restoration result may be set as an initial condition of an N-th image restoration result.

The microwave signal decomposition step may include the step of generating a plurality of first microwave signals by decomposing a measured microwave signal for each of a plurality of frequency components by wavelet conversion, and the step of generating a plurality of second microwave signals by decomposing a precalculated microwave signal for each of a plurality of frequency components by the wavelet conversion.

The method may further include after the microwave signal decomposing step, a breast cancer determining step of determining that the breast cancer exists at a portion that shows a value equal to or more than the threshold value in the plurality of first microwave signals.

In the breast cancer determining step, the breast cancer may be automatically determined through a look-up table prestoring for each frequency component a threshold value to determine that the tumor exists in the breast based on a value acquired by decomposing a microwave signal measured in a breast without the tumor for each frequency component.

According to embodiment of the present invention, a measured microwave signal is decomposed for each frequency component and a signal component generated by a small-sized tumor is decomposed based on the decomposed microwave signal to facilitate breast cancer detection from the decomposed microwave signal component.

A weight is applied to the microwave signal component generated by the small-sized tumor to restore a tomography image, thereby increasing imaging precision.

### BRIEF DESCRIPTION OF THE DRAWINGS

FIG. 1 is a conceptual diagram for describing a general microwave signal sensing method in an apparatus for breast cancer detection using a microwave.

FIG. 2 is a block diagram for describing a microwave signal tomography imaging method in the related art in the apparatus for breast cancer detection using a microwave.

FIG. 3 is a graph showing an example of a microwave signal measured on the circumference of a breast with respect to one transmission signal under a condition illustrated in FIG. 1.

FIG. 4 is a schematic block diagram of an apparatus for breast cancer detection according to an embodiment of the present invention.

FIG. 5 is a graph showing an example of decomposing a microwave signal measured in the case of a breast without the tumor in FIG. 3 according to the exemplar embodiment of the present invention.

FIG. 6 is a graph showing an example of decomposing a microwave signal measured in the case of a breast with a tumor in FIG. 3 according to the embodiment of the present invention.

FIG. 7 is a graph is a graph illustrating an example of a synthesized microwave signal generated by applying a predetermined weight to each of the decomposed microwave signals a₃, d₃, d₂, and d₁ illustrated in FIG. 6 according to the embodiment of the present invention.

FIG. 8 is a schematic block diagram of an apparatus for breast cancer detection according to an embodiment of the present invention.

FIG. 9 is a block diagram illustrating a microwave signal decomposing unit according to another embodiment of the present invention.

FIG. 10 is a detailed block diagram regarding a processing and imaging method of a microwave signal in an apparatus for breast cancer detection according to another embodiment of the present invention.

FIG. 11 is a flowchart of a method for breast cancer detection according to an embodiment of the present invention.

FIG. 12 is a flowchart of a method for breast cancer detection according to another embodiment of the present invention.

### DETAILED DESCRIPTION OF THE EMBODIMENTS

The aforementioned objects, characteristics, and advantages will be described below with reference to the accompanying drawings, and thus those skilled in the art to which the present invention pertains will easily implement the technical spirit of the present invention. In describing the present invention, when it is determined that the detailed description of the publicly known art related to the present invention may obscure the gist of the present invention, the detailed description thereof will be omitted. Further, respective terms described hereinbelow are just used for assisting understanding of the present invention and it should be noted that even though the respective terms are used for the same purpose, the respective terms may be used different terms in each manufacturing company or a research group. Hereinafter, embodiments according to the present invention will be described in detail with reference to the accompanying drawings.

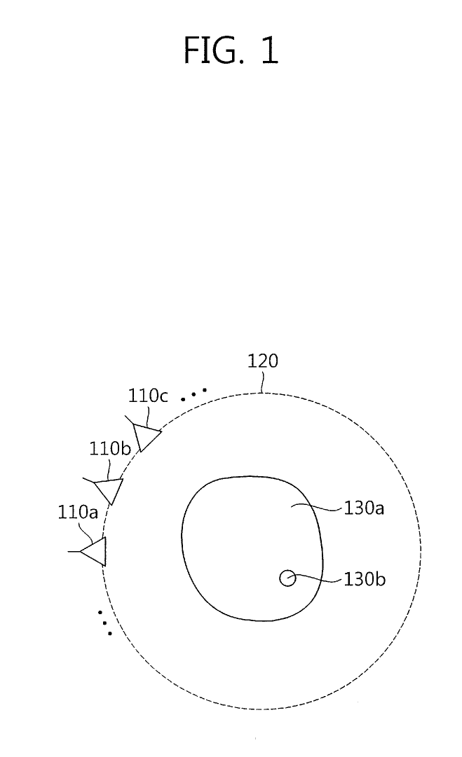

FIG. 1 is a schematic diagram for describing a general method of sensing a microwave signal in an apparatus for breast cancer detection using a microwave signal. A plurality of antennas 110a, 110b, and 110c is disposed around a breast 130 which is a cancer screening object, and a microwave scattering signal due to the breast is measured through a series of processes in which one antenna transmits a microwave and the remaining antennas receive the microwave. Alternatively, any one antenna around the breast transmits the microwave and the other antenna may also measure the microwave scattering signal due to the breast along the circumference of the breast.

Hereinafter, the microwave scattering signal due to the breast is referred to as the measured microwave.

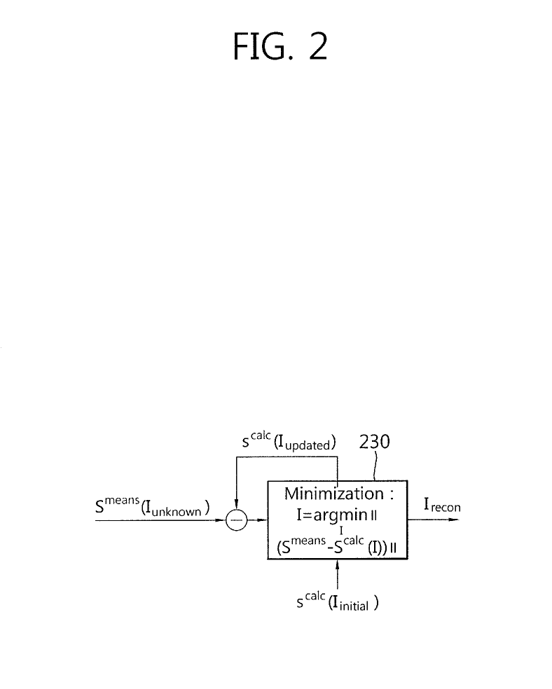

FIG. 2 is a schematic diagram for describing a method for microwave tomography in the related art in an apparatus for breast cancer detection using a microwave signal. Here, S^{means} is the measured microwave signal described in FIG. 1, I_{unknown} permittivity and conductivity information (image) in the actual breast to be restored, and S^{calc} is a microwave signal calculated in images Iᵢₙᵢₜᵢₐₗ to I_{updated} estimated in initial to intermediate steps. In addition, in order to minimize a difference between the microwave signal S^{means} measured in a state where the actual breast exists, that is, a unknown breast internal information I_{unknown} and the microwave signal S^{calc} calculated from the estimated breast internal information, an optimal breast internal permittivity/conductivity information is found. The found optimal breast internal permittivity/conductivity information corresponds to a tomography I_{recon} of a finally restored microwave signal.

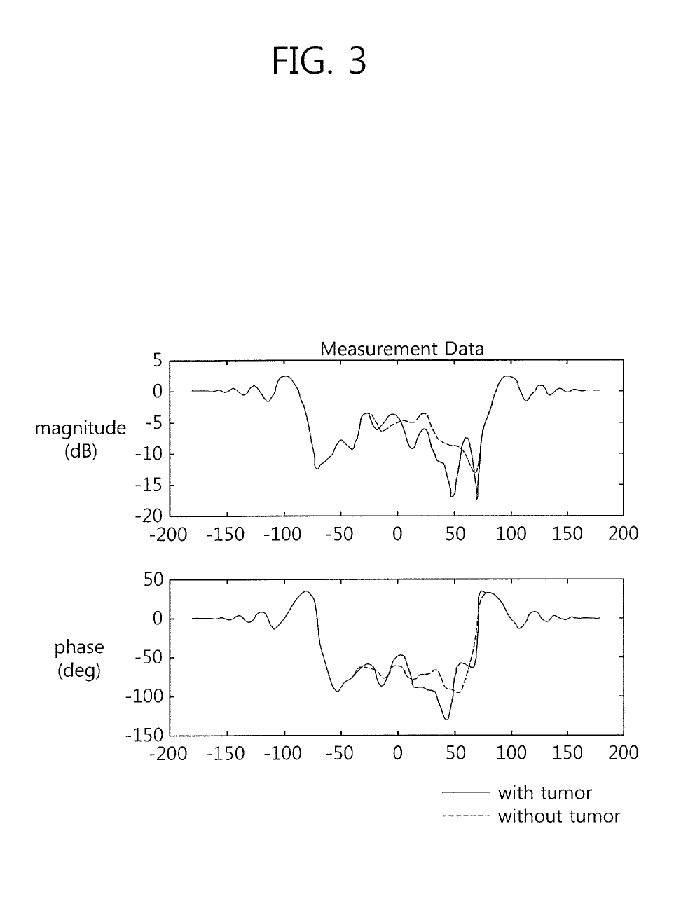

Meanwhile, FIG. 3 is a graph illustrating an example of the microwave signal measured along the breast circumference with respect to one transmitting signal under the same condition as FIG. 1. Here, the microwave signal is expressed by amplitude and a phase. Here, a signal illustrated by a dotted line is a case where there is no cancer in the breast, and a solid line is a case where there is a cancer in the breast. As illustrated in FIG. 3, it may be shown that a difference between signals is generated near 50 degrees of the measuring position. The difference between the signals is caused due to the tumor and is slightly shown as the tumor is smaller. Accordingly, unlike the apparatus for breast cancer detection that decomposes the measured microwave signal into a plurality of microwave signals for each frequency to detect the breast cancer according to predetermined values of a size and a phase of the decomposed microwave signal, in the related art described in FIG. 2, when the image is restored by a method of minimizing a difference between the entire measured microwave signal and the entire calculated microwave signal, it is difficult to discover the tumor, and particularly, it is more difficult to discover the small-sized tumor.

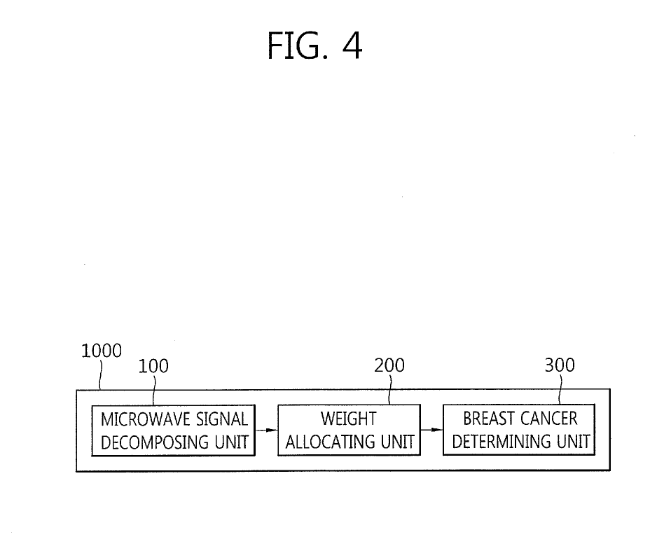

Referring to FIG. 4, an apparatus for breast cancer detection 1000 according to an embodiment of the present invention may include a microwave signal decomposing unit 100 and a breast cancer determining unit 300.

The microwave signal decomposing unit 100 according to another embodiment of the present invention decomposes the measured microwave signal for each frequency component to decompose the measured microwave signal into a plurality of frequency components from low-frequency components to high-frequency components. Preferably, the microwave signal decomposing unit 100 according to the embodiment of the present invention may decompose amplitude or phase data of the microwave signal through wavelet transform into the plurality of frequency components from low-frequency components to high-frequency components. Here, the microwave signal decomposing unit 100 performs the wavelet transform with respect to the measured microwave signal to decompose the amplitude and/or the phase data of the measured microwave signal into the plurality of frequency components from low-frequency components to high-frequency components.

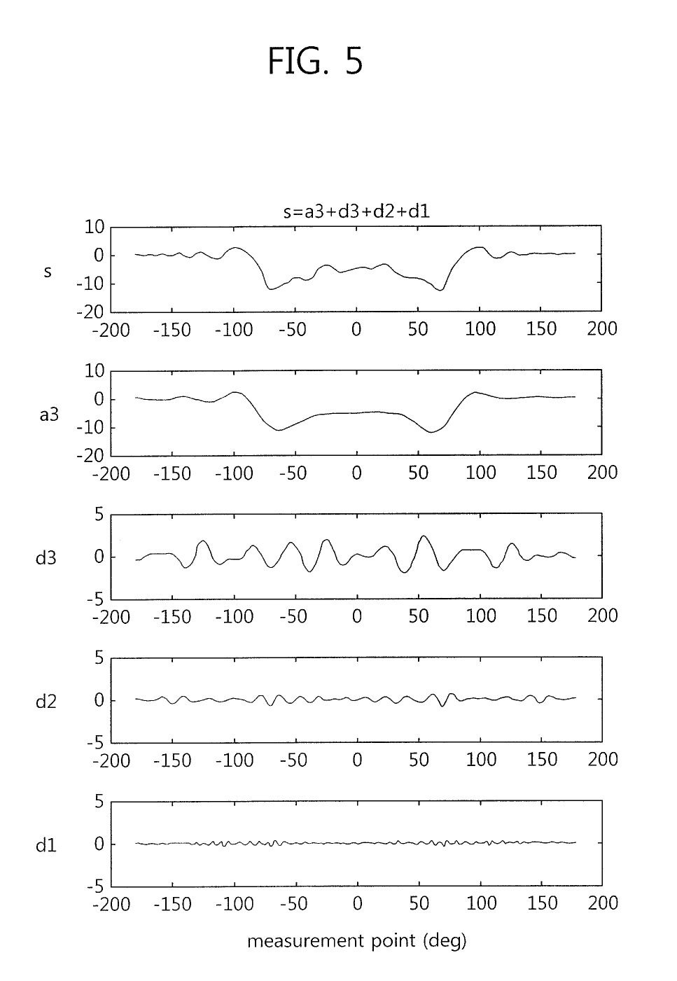

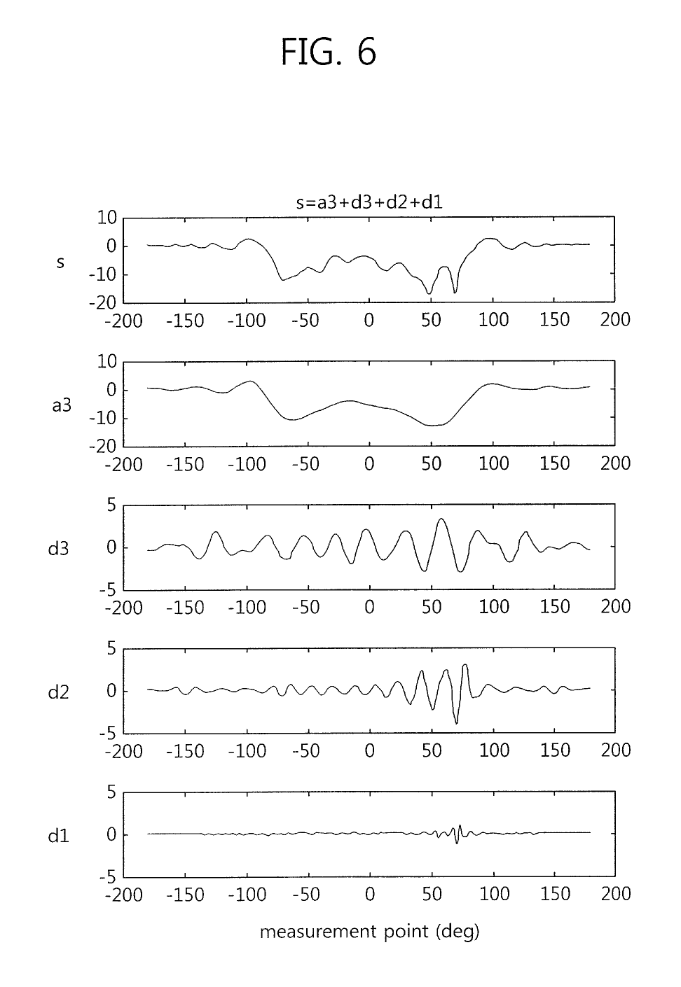

FIG. 5 is an exemplary diagram illustrating that the measured microwave signal is decomposed for each frequency component in the case of the breast without the tumor in FIG. 3. FIG. 6 is an exemplary diagram illustrating that the measured microwave signal is decomposed for each frequency component in the case of the breast with the tumor in FIG. 3. Referring to FIGS. 5 and 6, a sum of microwave signals a₃, d₃, d₂, and d₁ decomposed for each frequency component becomes an original signal s.

When comparing the decomposed microwave signals of FIGS. 5 and 6, particularly, D₂ to d₁ are different from each other. According to the embodiment of the present invention, in the signal around 50 degrees of d₂ to d₁, the microwave signal of the tumor like FIG. 6 is larger than the microwave signal of no tumor like FIG. 5. The difference is caused by the existence of the tumor. Accordingly, according to the embodiment of the present invention, a microwave signal component generated by a small tumor is decomposed for each frequency component to easily determine whether the tumor exists directly from the decomposed microwave signal.

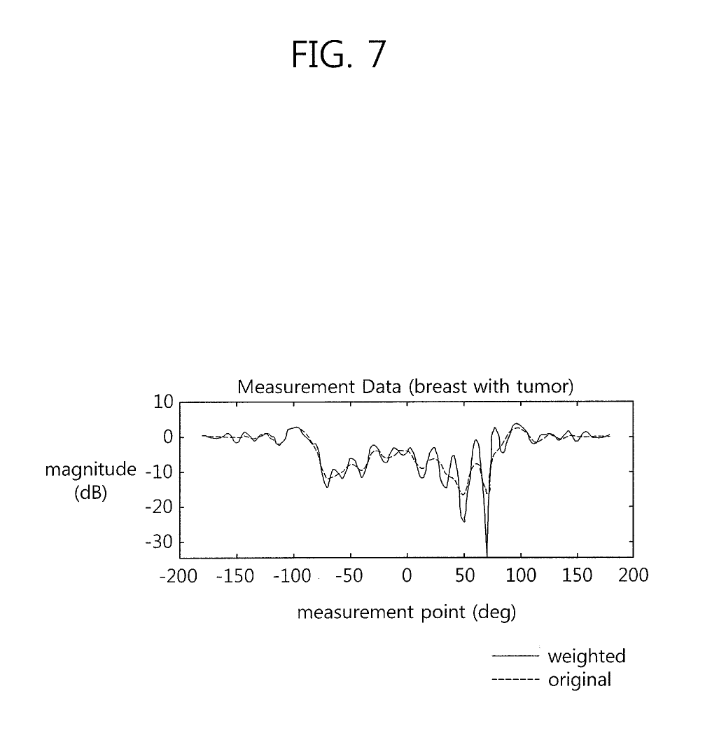

Referring to FIG. 4, the apparatus 1000 for breast cancer detection according to the embodiment of the present invention may further include a weight allocating unit 300. FIG. 7 is a graph illustrating an example of a synthesized microwave signal generated by applying a predetermined weight to each of the decomposed microwave signals a₃, d₃, d₂, and d₁ illustrated in FIG. 6 according to the embodiment of the present invention.

Referring to FIG. 7, a signal component contributed by the tumor may be amplified by applying the predetermined weight to the microwave signals a₃, d₃, d₂, and d₁ decomposed into the plurality of frequency components. FIG. 7 is an exemplary diagram of a newly synthesized microwave signal by applying the predetermined weight to each of the decomposed microwave signals a₃, d₃, d₂, and d₁ illustrated in FIG. 6. Referring to FIG. 7, a signal near 50 degrees of the measured position where the tumor exists may be more amplified than other signal.

The breast cancer determining unit 200 according to the embodiment of the present invention may determine the breast cancer based on the microwave signal decomposed into the plurality of frequency components. The breast cancer determining unit 200 may determine that the breast cancer exists at a portion having a value of a threshold value or more of the microwave signal decomposed into the plurality of frequency components.

For example, the threshold value may be a value that may be determined as the tumor based on the pre-determined microwave signals for each frequency in the breast without the tumor (a normal breast). Alternatively, the threshold value may be a value that may be determined as the tumor based on the values obtained by applying the predetermined weight to the pre-determined microwave signals for each frequency in the breast without the tumor (the normal breast).

Alternatively, the breast cancer determining unit 200 according to the embodiment of the present invention may further include a look-up table unit (not illustrated). The look-up table unit may pre-store the threshold value that may be determined as the tumor in the breast, based on the values obtained by decomposing the microwave signal measured in the breast without the tumor (the normal breast) for each frequency component to automatically detect the breast cancer. Similarly, the threshold value may be a value that may be determined as the tumor based on the pre-determined microwave signals for each frequency in the breast without the tumor (the normal breast).

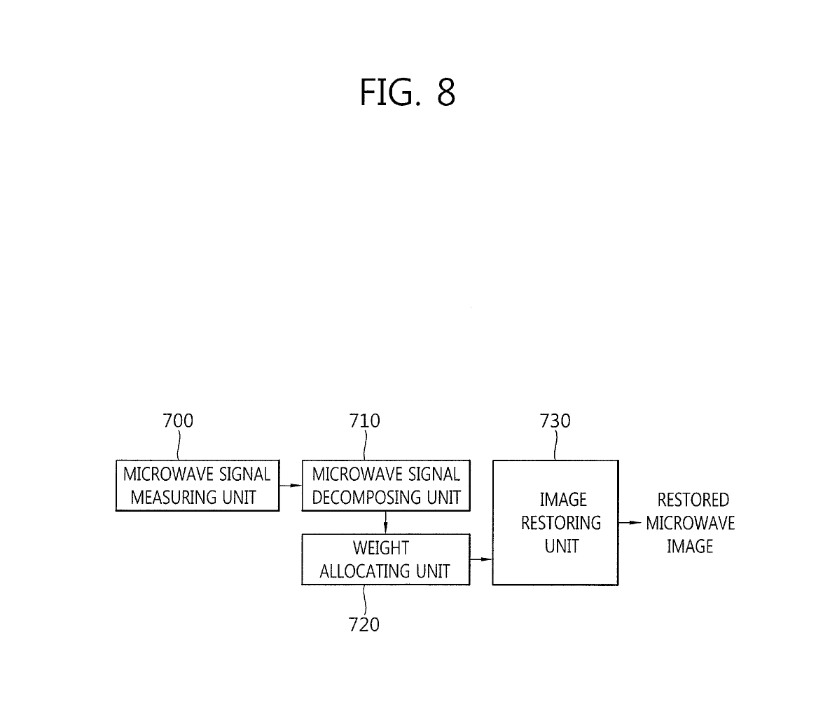

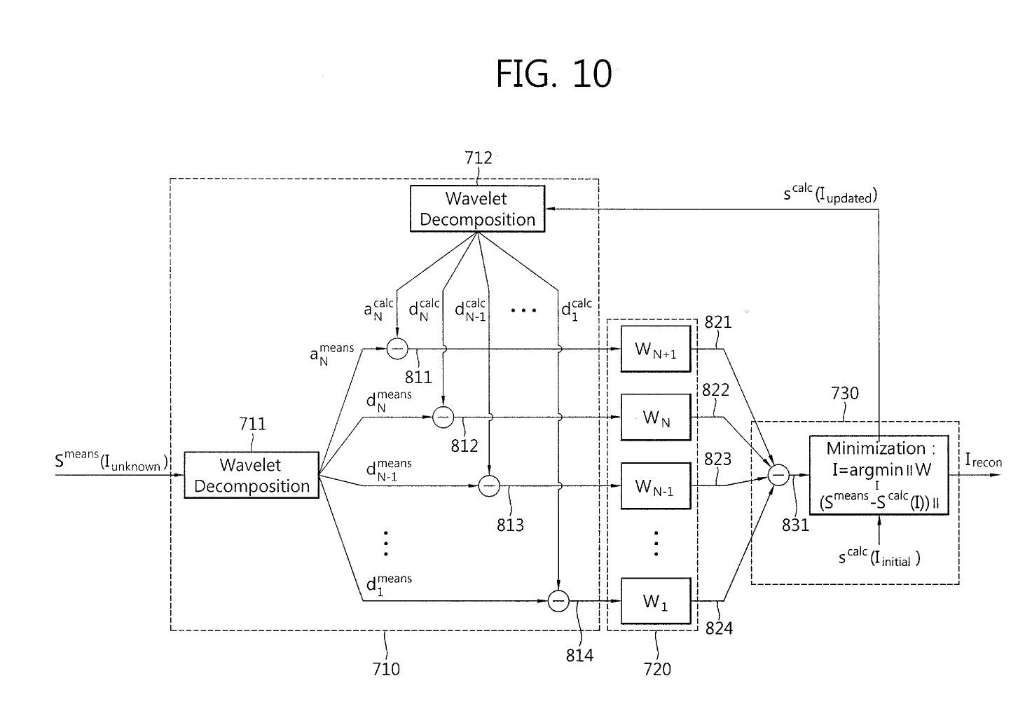

FIG. 8 is a block diagram of an apparatus for breast cancer detection using a microwave signal according to another embodiment of the present invention, and FIG. 10 is a detailed block diagram for a method of processing and imaging a microwave signal measured in an apparatus 1000 for breast cancer detection according to another embodiment of the present invention. Referring to FIGS. 8 and 10, the apparatus 1000 for breast cancer detection according to another embodiment of the present invention may include a microwave signal measuring unit 700, a microwave signal decomposing unit 710, a weight allocating unit 720, and an image restoring unit 730.

The microwave signal measuring unit 700 according to another embodiment of the present invention may include a plurality of antennas to transmit and receive the microwave signal. The transmitting antenna transmits the microwave signal to a breast which is an object to be detected. The transmitted microwave signal is scattered by the breast and received by other antennas surrounding the breast (alternatively, a separate receiving antenna moves along the circumference of the breast). The received microwave signal may be measured as data having amplitude and phase information in a complex number form by a separate measuring unit or measured as data having amplitude and phase information in a complex number form in the antenna receiving the microwave signal.

The microwave signal decomposing unit 710 according to another embodiment of the present invention decomposes the measured microwave signal measured by the microwave signal measuring unit 700 for each frequency component to decompose the measured microwave signal into a plurality of frequency components from low-frequency components to high-frequency components. Preferably, the microwave signal decomposing unit 710 according to another embodiment of the present invention may decompose amplitude or phase data of the microwave signal through wavelet transform into the plurality of frequency components from low-frequency components to high-frequency components.

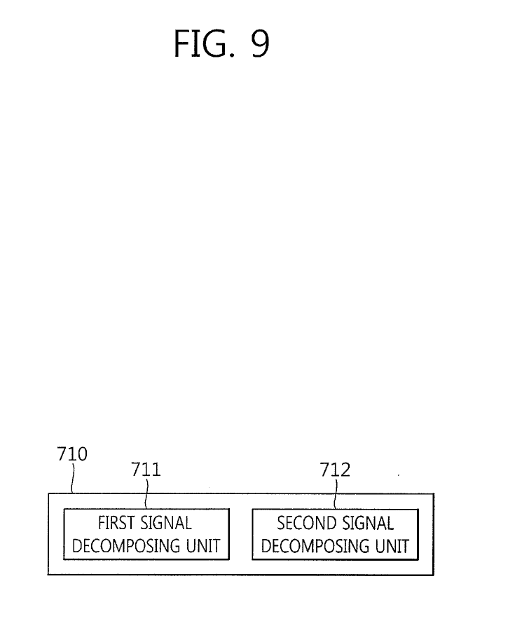

FIG. 9 is a block diagram illustrating the microwave signal decomposing unit 710 according to another embodiment of the present invention. Referring to FIG. 9, the microwave signal decomposing unit 710 may include a first signal decomposing unit 711 and a second signal decomposing unit 712. The first signal decomposing unit 711 may decompose a measured microwave signal S^{means} into a plurality of components to generate a plurality of first microwave signals a_{N}^{means}, d_{N}^{means}, d_{N-1}^{means}, . . . d₁^{means}. The second signal decomposing unit 712 may decompose a microwave signal calculated from images Iᵢₙᵢₜᵢₐₗ to I_{updated} estimated in initial to intermediate steps into the plurality of components to generate a plurality of second microwave signals a_{N}^{calc}, d_{N}^{calc}, d_{N-1}^{calc}, . . . , d₁^{calc}. Here, the first signal decomposing unit 711 and the second signal decomposing unit 712 may decompose the measured microwave signal S^{means} into a plurality of components a_{N}^{means}, d_{N}^{means}, d_{N-1}^{means, . . . , d}₁^{means} by using wavelet transform or decompose the microwave signal calculated from the images Iᵢₙᵢₜᵢₐₗ to I_{updated} estimated in initial to intermediate steps into the plurality of components a_{N}^{calc}, d_{N}^{calc}, d_{N-1}^{calc}, d₁^{calc}.

Referring to FIGS. 8 to 10, the weight allocating unit 720 according to another embodiment of the present invention may calculate differences between the plurality of first microwave signals a_{N}^{means}, d_{N}^{means}, d_{N-1}^{means}, . . . , d₁^{means} and the plurality of second microwave signals a_{N}^{calc}, d_{N}^{calc}, d_{N-1}^{calc}, . . . , d₁^{calc} to generate a plurality of differential microwave signals 811, 812, 813, and 814, and apply at least one weight to each of the plurality of differential microwave signals 811, 812, 813, and 814 to generate one or more weight microwave signals 821, 822, 823, and 824.

The image restoring unit 730 according to another embodiment of the present invention may restore a tomography of the breast by applying a predetermined image restoration algorithm to the weight microwave signals 821, 822, 823, and 824.

Referring back to FIG. 10, in the apparatus for breast cancer detection according to another embodiment of the present invention, the weight allocating unit 720 may generate the plurality of weight microwave signals 821, 822, 823, and 824 by applying the weight to all of the plurality of differential microwave signals 811, 812, 813, and 814, the image restoring unit 730 may generate a synthesized microwave signal 831 by combining the weight microwave signals 821, 822, 823, and 824 and find an optimal image I_{recon} while updating the image (permittivity to conductivity in the breast) so that the synthesized microwave signal 831 is the minimum.

Alternatively, according to another embodiment of the weight allocating unit 720 and the image restoring unit 730 according to another embodiment of the present invention, the weight allocating unit 720 may generate the plurality of weight microwave signals 821, 822, 823, and 824 by applying the weight to all of the plurality of differential microwave signals 811, 812, 813, and 814 and generate the synthesized microwave signal 831 by combining the plurality of weight microwave signals 821, 822, 823, and 824, and the image restoring unit 730 may find the optimal image I_{recon} while updating the image (permittivity to conductivity in the breast) so that the synthesized microwave signal 831 is the minimum.

Further, in another embodiment of the image restoring unit 730 according to another embodiment of the present invention, the image restoring unit 730 may perform the image restoration N times in sequence, applies first to N-th weights to the plurality of differential microwave signals during the N-th image restoration, perform the image restoration by setting the remaining weights to 0, and set the N−1-th image restoration result as an initial condition of the N-th image restoration result.

For example, the image restoring unit 730 may perform the first image restoration by applying the weight w_{N+1} to the first differential microwave signal 811 while setting the remaining weights except for w_{N+1} to 0 during the first image restoration. During the second image restoration, while the remaining weights except for w_{N+1} and w_{N} are set to 0 and the first image restoration result is set to the initial condition Iᵢₙᵢₜᵢₐₗ, the image restoring unit 730 may perform the second image restoration by applying the weight w_{N+1} to the first differential microwave signal 811 and applying the weight w_{N} to the second differential microwave signal 812. During the third image restoration, while the remaining weights except for w_{N+1}, w_{N}, and w_{N-1} are set to 0 and the second image restoration result is set to the initial condition Iᵢₙᵢₜᵢₐₗ, the image restoring unit 730 may perform the third image restoration by applying the weight w_{N+1} to the first differential microwave signal 811, applying the weight wN to the second differential microwave signal 812, and applying the weight w_{N-1} to the third differential microwave signal 813, and as a result, a series of sequential image restorations may be performed.

The apparatus 1000 for breast cancer detection according to the embodiment of the present invention may further include a breast cancer determining unit (not illustrated). The breast cancer determining unit (not illustrated) according to the embodiment of the present invention may determine that the breast cancer exists at a portion having a value of a threshold value or more in at least one first microwave signal among the plurality of first microwave signals decomposed for each frequency component. For example, the threshold value may be a value that may be determined as the tumor based on the pre-determined microwave signals for each frequency in the breast without the tumor (a normal breast). Alternatively, the threshold value may be a value that may be determined as the tumor based on the values obtained by applying the predetermined weight to the pre-determined microwave signals for each frequency in the breast without the tumor (the normal breast). Alternatively, the threshold value may be a value that may be determined as the tumor based on a differential value between the plurality of second microwave signals a_{N}^{calc}, d_{N}^{calc}, D_{N-1}^{calc}, . . . , d₁^{calc} and the plurality of first microwave signals a_{N}^{means}, d_{N}^{means}, d_{N-1}^{means}, . . . , d₁^{means}.

Alternatively, the breast cancer determining unit (not illustrated) according to the embodiment of the present invention may further include a look-up table unit (not illustrated). The look-up table unit may pre-store the threshold value that may be determined as the tumor in the breast, based on the values obtained by decomposing the microwave signal measured in the breast without the tumor (the normal breast) for each frequency component to automatically detect the breast cancer. Similarly, the threshold value may be a value that may be determined as the tumor based on the pre-determined microwave signals for each frequency in the breast without the tumor (the normal breast). Alternatively, the threshold value may be a value that may be determined as the tumor based on a differential value between the plurality of second microwave signals and the plurality of first microwave signals.

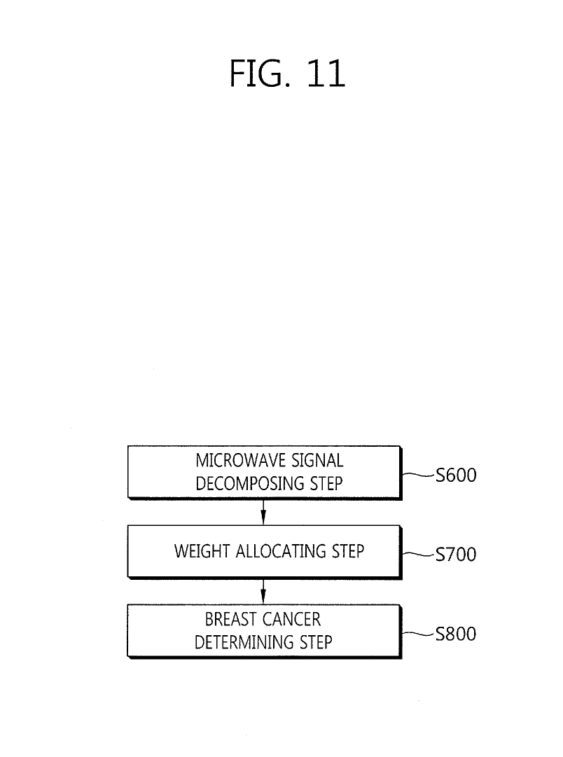

FIG. 11 is a flowchart of a method for breast cancer detection according to an embodiment of the present invention. Referring to FIG. 11, the method for breast cancer detection according to the embodiment of the present invention may include a microwave signal decomposition step (S600) of decomposing a microwave signal measured on the circumference of a breast for each of a plurality of frequency components and the step (S800) of determining a breast cancer based on the microwave signal decomposed for each of the plurality of frequency components. According to the embodiment of the present invention, in the microwave signal decomposition step (S600), the measured microwave signal is wavelet-converted to decompose the measured microwave signal for each of the plurality of frequency components.

The method for breast cancer detection according to the embodiment of the present invention may further include a weight allocating step (S700) of applying at least one weight to the microwave signal decomposed after the microwave signal decomposition step (S600).

Referring to FIG. 11, in the step of determining the breast cancer according to the embodiment of the present invention, it may be determined that the breast cancer exists at a portion showing a threshold value or more among the microwave signals (based on the measured microwave signal) a₃, d₃, d₂, and d₁ decomposed for each of the plurality of frequency components. For example, the threshold value may be a value to determine that there is a tumor based on premeasured microwave signals for each frequency in the breast without the tumor (normal breast). Alternatively, the threshold value may be a value to determine that there is a tumor based on values to apply a predetermined weight to the premeasured microwave signals for each frequency in the breast without the tumor (normal breast).

Alternatively, the step of determining the breast cancer according to the embodiment of the present invention may further includes a look-up table unit (not illustrated). The look-up table unit previously stores the threshold value to determine that the tumor exists in the breast for each frequency component to automatically detect the breast caner based on the values acquired by decomposing the microwave signal measured in the breast without the tumor (normal breast) for each frequency component. Similarly as above, the threshold value may be a value to determine that there is the tumor based on the premeasured microwave signals for each frequency in the breast without the tumor (normal breast).

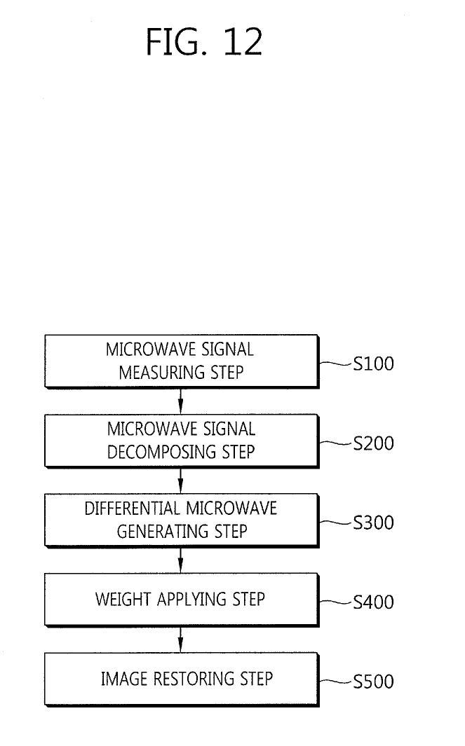

FIG. 12 is a flowchart of a method for breast cancer detection according to another embodiment of the present invention. Referring to FIG. 12, the method for breast cancer detection according to another embodiment of the present invention may include the step (S100) of measuring a microwave signal S^{means}, a microwave signal decomposing step (S200), the step (S300) of generating a plurality of differential microwave signals, a weight applying step (S400), and an image restoring step (S500).

In the step (S100) of measuring the microwave signal according to another embodiment of the present invention, the microwave signal is measured on the circumference of the breast. In this step, a plurality of antennas may be provided to transmit and receive the microwave signal. The transmission antenna transmits the microwave signal to the breast which is an objected to be diagnosed. The transmitted microwave signal is scattered by the breast to be received by other antennas (alternatively, a separate transmission antenna moves around the breast) surrounding the breast. The received microwave signal may be measured as data having complex number type amplitude and phase information by a separate measurement unit or measured as the complex number type amplitude and phase information in the antenna that receives the microwave signal.

The microwave signal decomposing step (S200) according to another embodiment of the present invention may include a first signal decomposing step of decomposing the measured microwave signal S^{means} for each of the plurality of frequency components to generate a plurality of first microwave signals a_{N}^{means}, d_{N}^{means}, d_{N-1}^{means}, . . . , d₁^{means} and a second signal decomposing step of decomposing a precalculated microwave signal (for example, a microwave signal calculated in an estimated image in an initial step or an intermediate step) for each of the plurality of frequency components to generate a plurality of second microwave signals a_{N}^{calc}, d_{N}^{calc}, d_{N-1}^{calc}, . . . , d₁^{calc}. In the microwave signal decomposing step (S200) according to another embodiment of the present invention, the plurality of first microwave signals a_{N}^{means}, d_{N}^{means}, d_{N-1}^{means}, . . . , d₁^{means} and the plurality of second microwave signals a_{N}^{calc}, d_{N}^{calc}, d_{N-1}^{calc}, . . . , d₁^{calc} may be generated by using wavelet conversion. In the step (S300) of generating a plurality of differential microwave signals according to another embodiment of the present invention, differences of the plurality of first microwave signals a_{N}^{means}, d_{N}^{means}, d_{N-1}^{means}, . . . , d₁^{means} and the plurality of second microwave signals a_{N}^{calc}, d_{N}^{calc}, d_{N-1}^{calc}, . . . , d₁^{calc} are acquired to generate a plurality of differential microwave signals 811, 812, 813, and 814. For example, in FIG. 10, a_{N}^{calc} and a_{N}^{means} may have the same frequency component and the differential microwave signal 811 may a_{N}^{calc}-a_{N}^{means} and d_{N}^{calc} and d_{N}^{means} may have the same frequency component and the differential microwave signal 812 may be d_{N}^{calc}-d_{N}^{means}.

In the weight applying step (S400) according to another embodiment of the present invention, weights w_{N+1}, W_{N}, w_{N-1}, . . . , w₁ are applied to at least one of the plurality of differential microwave signals 811, 812, 813, and 814 to generate at least one weight microwave signal 821, 822, 823, and 824.

In the image restoring step (S500) according to another embodiment of the present invention, the tomography image of the breast may be restored by using at least one weight microwave signal 821, 822, 823, and 824 provided from the weight applying step (S400).

Continuously, referring to FIG. 10, in the method for breast cancer detection according to another embodiment of the present invention, in the weight applying step (S400), the weights are applied to all of the plurality of differential microwave signals 811, 812, 813, and 814 to generate the plurality of weight microwave signal 821, 822, 823, and 824 and in the image restoring step (S500), the plurality of weight microwave signals 821, 822, 823, and 824 are combined to generate a synthesized microwave signal 831 and update an image (dielectric constant or conductivity in the breast) so that the synthesized microwave signal 831 is minimum.

Alternatively, in the weight applying step (S400), the weights are applied to all of the plurality of differential microwave signals 811, 812, 813, and 814 to generate the plurality of weight microwave signal 821, 822, 823, and 824 and the plurality of weight microwave signals 821, 822, 823, and 824 are combined to generate the synthesized microwave signal 831 and in the image restoring step (S500), the image (dielectric constant or conductivity in the breast) may be updated so that the synthesized microwave signal 831 is minimum.

Alternatively, in another embodiment of the image restoring step (S500) according to another embodiment of the present invention, image restoration is sequentially performed N times and in N-th image restoration, first to N-th weights are applied to the plurality of differential microwave signals and the image restoration is performed by setting a remaining weight to 0 and an N−1-th image restoration result may be set as an initial condition of an N-th image restoration result. For example, in the state where remaining weights other than w_{N+1} are set to 0 in the first image restoration, the weight w_{N+1} is applied to the first differential microwave signal 811 to the first image restoration. In the second image restoration, remaining weights other than w_{N+1} and w_{N} are set to 0 and the first image restoration result is set as the initial condition Iᵢₙᵢₜᵢₐₗ, the weight w_{N+1} is applied to the first differential microwave signal 811 and the weight w_{N} is applied to the second differential microwave signal 812 to perform the second image restoration. In the third image restoration, remaining weights other than w_{N+1}, w_{N}, and w_{N-1} are set to 0 and a result of the first image restoration result is set as the initial condition Iᵢₙᵢₜᵢₐₗ, the weight w_{N+1} is applied to the first differential microwave signal 811, the weight W_{N} is applied to the second differential microwave signal 812, and the weight w_{N-1} is applied to the third differential microwave signal 813 to perform the third image restoration, as a result, a series of sequential image restoration may be performed according to the order.

The method for breast cancer detection according to another embodiment of the present invention may further include a breast cancer determining step (not illustrated) of determining that the breast cancer exists at a portion that shows a value equal to or more than the threshold value in the plurality of first microwave signals (a_{N}^{means}, d_{N}^{means}, d_{N-1}^{means}, . . . , d₁^{means}; based on the measured microwave signal) after the microwave signal decomposing step (S200). For example, the threshold value may be a value to determine that there is a tumor based on premeasured microwave signals for each frequency in the breast without the tumor (normal breast). Alternatively, the threshold value may be a value to determine that there is a tumor based on values to apply a predetermined weight to the premeasured microwave signals for each frequency in the breast without the tumor (normal breast). Alternatively, the threshold value may be a value to determining that there is the tumor based on difference values of the plurality of second microwave signals a_{N}^{calc}, d_{N}^{calc}, d_{N-1}^{calc}, . . . , d₁^{calc} and the first microwave signals a_{N}^{means}, d_{N}^{means}, d_{N-1}^{means}, . . . , d₁^{means}.

Alternatively, the breast cancer determining step (not illustrated) according to the embodiment of the present invention may further includes a look-up table unit (not illustrated). The look-up table unit previously stores the threshold value to determine that the tumor exists in the breast for each frequency component to automatically detect the breast caner based on the values acquired by decomposing the microwave signal measured in the breast (normal breast) without the tumor for each frequency component. Similarly as above, the threshold value may be a value to determine that there is the tumor based on the premeasured microwave signals for each frequency in the breast without the tumor (normal breast). Alternatively, the threshold value may be a value to determining that there is the tumor based on the difference values of the plurality of second microwave signals and the first microwave signals.

As described above, when an optimal image is found by decomposing the measured microwave signal for each of the plurality of frequency components or the image restoration is sequentially by decomposing the measured microwave signal for each of the plurality of frequency components, the image restoration of the present invention is more excellent than an image restoring method using all microwaves signals (microwave signals which are decomposed for each of the plurality of frequency components) in the related art. Accordingly, as compared with the breast cancer detecting method using all of the microwave signals in the related art, the small-sized tumor may be distinctively imaged and the breast cancer may be early diagnosed.

Although the embodiments of the present disclosure have been described with reference to the accompanying drawings as described above, those skilled in the art will be able to understand that the present disclosure can be implemented in other detailed forms without changing the technical spirit or an essential characteristic. For example, those skilled in the art may modify each component according to an application or combine or substitute disclosed embodiments to practice forms that are not disclosed in the embodiment of the present invention, which does not depart from the scope of the present invention. Therefore, since the embodiment of the present invention described above is only an example, it is not to be restrictively interpreted. In addition, these modified embodiments are to be included in the technical spirit of the present invention as disclosed in the following claims.

What is claimed is:

1. An apparatus for apparatus for breast cancer detection using a microwave signal, the apparatus comprising:
a microwave signal decomposing unit configured to decompose a microwave signal measured along a circumference of a breast into a plurality of frequency components; and
a beast cancer determining unit configured to determining the breast cancer based on the microwave signal decomposed into the plurality of frequency components.

2. The apparatus of claim 1, wherein the microwave signal decomposing unit decomposes the measured microwave signal into the plurality of frequency components by performing wavelet transform with respect to the measured microwave signal.

3. The apparatus of claim 1, wherein the breast cancer determination unit determines that the breast cancer exists at a portion having a value of a threshold value or more in the microwave signal decomposed into the plurality of frequency components.

4. The apparatus of claim 1, further comprising:
a weight allocating unit configured to apply at least one weight to the microwave signal decomposed into the plurality of frequency components.

5. An apparatus for apparatus for breast cancer detection using a microwave signal, the apparatus comprising:
a microwave signal measuring unit configured to measure the microwave signal along the circumference of the breast;
a microwave signal decomposing unit including a first signal decomposing unit decomposing the microwave signal measured from the microwave signal measuring unit into a plurality of frequency components to generate a plurality of first microwave signals and a second signal decomposing unit decomposing a pre-calculated microwave signal into a plurality of frequency components to generate a plurality of second microwave signals;
a weight allocating unit configured to calculate differences between the plurality of first microwave signals and the plurality of second microwave signals to generate a plurality of differential microwave signals and apply at least one weight to the plurality of differential microwave signals to generate one or more weight microwave signals; and
an image restoring unit configured to restore a tomography of the breast by using the microwave signal provided from the weight allocating unit.

6. The apparatus of claim 5, wherein:
the weight allocating unit generates the plurality of weight microwave signals by applying the weights to all of the plurality of differential microwave signals, and
the image restoring unit generates a synthesized microwave signal by combining the plurality of weight microwave signals and updates the image so that the synthesized microwave signal is the minimum.

7. The apparatus of claim 5, wherein:
the image restoring unit performs the image restoration N times in sequence,
during the N-th image restoration,
applies first to N-th weights to the plurality of differential microwave signals, performs the image restoration by setting the remaining weights to 0, and
sets the N−1-th image restoration result as an initial condition of the N-th image restoration result.

8. The apparatus of claim 5, further comprising:
a breast cancer determining unit configured to determine that the breast cancer exists at a portion having a value of a threshold value or more in the plurality of first microwave signals.

9. The apparatus of claim 8, wherein the breast cancer determining unit further includes a look-up table that pre-store the threshold value that is determined as the tumor in the breast, based on the values obtained by decomposing the microwave signal measured in the breast without the tumor for each frequency component.

10. A method for breast cancer detection using a microwave signal, the method comprising:
a microwave signal decomposition step of decomposing a microwave signal measured on the circumference of a breast for each of a plurality of frequency components; and
a breast cancer determining step of determining a breast cancer based on the microwave signal decomposed for each of the plurality of frequency components.

11. The method of claim 10, wherein:
in the microwave signal decomposition step
the measured microwave signal is wavelet-converted to decompose the measured microwave signal for each of the plurality of frequency components.

12. The method of claim 10, wherein in the step of determining the breast cancer, it is determined that the breast cancer exists at a portion showing a threshold value or more among the microwave signals decomposed for each of the plurality of frequency components.

13. The method of claim 10, further comprising:
a weight allocating step of applying the weights to the plurlaity of microwave signals decomposed for each of the plurality of frequency components to generate at least one microwave signal, after the microwave signal decomposition step.

14. A method for breast cancer detection using a microwave signal, the method comprising:
measuring a microwave signal on the circumference of a breast;
a microwave signal decomposing step including a first signal decomposing step of decomposing the measured microwave signal for each of the plurality of frequency components to generate a plurality of first microwave signals and a second signal decomposing step of decomposing a precalculated microwave signal for each of the plurality of frequency components to generate a plurality of second microwave signals;
a differential microwave generating step of acquiring differences of the plurality of first microwave signals and the plurality of second microwave to generate a plurality of differential microwave signals;
a weight applying step of applying weights to at least one of the plurality of differential microwave signals to generate at least one weight microwave signal; and
an image restoring step of restoring the tomography image of the breast by using at least one weight microwave signal provided from the weight applying step.

15. The method of claim 14, wherein the precalculated microwave signal is a microwave signal calculated in an estimated image in an initial step to an intermediate step.

16. The method of claim 14, wherein:
in the weight applying step, the weights are applied to all of the plurality of differential microwave signals to generate the plurality of weight microwave signal, and
in the image restoring step, the plurality of weight microwave signals are combined to generate a synthesized microwave signal and update an image so that the synthesized microwave signal is minimum.

17. The method of claim 14, wherein:
in the image restoring step, image restoration is sequentially performed N times, and
in N-th image restoration,
first to N-th weights are applied to the plurality of differential microwave signals and the image restoration is performed by setting a remaining weight to 0, and
an N−1-th image restoration result is set as an initial condition of an N-th image restoration result.

18. The method of claim 14, wherein:
the microwave signal decomposition step includes
the step of generating a plurality of first microwave signals by decomposing a measured microwave signal for each of a plurality of frequency components by wavelet conversion, and
the step of generating a plurality of second microwave signals by decomposing a precalculated microwave signal for each of a plurality of frequency components by the wavelet conversion.

19. The method of claim 13, further comprising:
after the microwave signal decomposing step,
a breast cancer determining step of determining that the breast cancer exists at a portion that shows a value equal to or more than the threshold value in the plurality of first microwave signals.

20. The method of claim 18, wherein:
in the breast cancer determining step,
the breast cancer is automatically determined through a look-up table prestoring for each frequency component a threshold value to determine that the tumor exists in the breast based on a value acquired by decomposing a microwave signal measured in a breast without the tumor for each frequency component.
